(19) United States
(12) Patent Application Publication
(10) Pub. No.: US 2018/0177560 A1
(43) Pub. Date: Jun. 28, 2018

(54) STERILE CONTAINER, COMPRISING A PLURALITY OF CENTRALLY OPERATED LOCKING ELEMENTS

(71) Applicant: Aesculap AG, Tuttlingen (DE)

(72) Inventor: Dieter WEISSHAUPT, Immendingen (DE)

(21) Appl. No.: 15/739,224

(22) PCT Filed: Jun. 9, 2016
(86) PCT No.: PCT/EP2016/063204

§ 371 Date: Dec. 22, 2017

(30) Foreign Application Priority Data
Jun. 29, 2015 (DE) 10 2015 110 419.9

(51) Int. Cl. A61B 50/30; B65D 45/16; B65D 47/32

(52) U.S. Cl. CPC A61B 50/30; A61B 2050/006; B65D 47/32; B65D 45/16

(57) ABSTRACT

A sterile container includes a plurality of locking elements that can be operated via at least one operating strip by at least one common operating lever. This allows an active discharge of condensate and controlled pressure compensation in a reliable manner and offers more safety when storing and transporting the sterile container.

### RELATED APPLICATIONS

This application is the United States national phase entry of International Application No. PCT/EP2016/063204, filed Jun. 9, 2016, which is related to and claims the benefit of priority of German Application No. 10 2015 110 419.9, filed Jun. 29, 2015. The contents of International Application No. PCT/EP2016/063204 and German Application No. 10 2015 110 419.9 are incorporated by reference herein in their entireties.

### FIELD

The present invention relates to a sterile container comprising a plurality of centrally operated locking elements and especially to a sterile container comprising a plurality of locking elements that can be operated via an operating strip by at least one common operating lever, with the number of operating elements being smaller than the number of locking elements operable by the latter.

### BACKGROUND

For the vast majority of clinical activities and operations it is indispensable to ensure sterility of the instruments used and/or of any other tools. For this reason, sterile containers (also referred to as sterilization containers) that are equipped/filled with medical instruments, for example, are used.

Then the sterilization container is heated in the equipped/filled state for instance in an autoclave for a predetermined period of time to a predetermined sterilization temperature, until possible microorganisms adhering to the medical instruments have been killed.

During the sterilization process, for example in an autoclave, condensate is formed inside the sterile container. In order to remove said condensate from the sterile container, in container systems including active discharge of condensate the difference in pressure between the positive pressure prevailing in the sterile container and the negative pressure prevailing in the sterilization chamber is used to discharge the condensate.

Conventional sterile containers usually include a cover or a door attachable to a container trough as well as one or two locking elements by which the cover or the door is kept closed and locked on the sterile container. In such conventional sterile containers, during the active discharge of condensate deformations of the container trough and, resp., of the container body and the cover of the container may occur due to the pressure difference between the inside of the sterile container and the sterilization chamber which deformations may allow the excess pressure to escape or pressure compensation to take place.

However, such deformations result in the fact that after such uncontrolled pressure compensation there will not be sufficient pressure remaining in the sterile container for successfully discharging all of the condensate contained in the sterile container. This will result in undesired residual moisture inside the sterile container.

### SUMMARY

The object underlying the present invention is to provide a sterile container comprising active discharge of condensate in which deformations of the sterile container due to pressure differences are largely avoided or will not result in any pressure loss.

A sterile container according to the invention comprising active discharge of condensate includes a plurality of operable locking elements which can be or are operated via (at least) one operating strip by means of (at least) one common operating lever. I.e. plural locking elements or locking units (spaced apart from the container periphery) are (functionally) combined into one locking mechanism which can be jointly actuated by means of a (manual) operating device or an operating unit (independently of possibly further locking mechanisms of this structure). The operating effort thus is equal to that of the known prior art, whereas the closing safety is improved by virtue of the arrangement of plural locking units jointly/centrally operated by one operating unit.

Consequently, the key idea resides in the fact that the plural locking elements spaced apart from each other on the container periphery (also referred to as the container circumference) or spaced apart from the opening offer a stable, because multi-point, connection between the container cover/door and the container trough of the sterile container, which increases the resisting force of the sterile container against deformations. In this way, leakages due to deformations occurring during the sterilization process are avoided and the pressure compensation between the interior of the sterile container and the sterilization chamber may be utilized for discharging the condensate formed in the sterilization container in a controlled and efficient manner.

In other words, at the container trough and at the container cover a plurality of pairs of locking elements tightly arranged thereon are located of which at least one locking element of each pair of locking elements is movably held to be non-detachable on the container trough or on the container cover and can be brought into locking engagement with another locking element of the same pair. The at least one preferably flexible operating strip (also referred to as an actuation strip) includes engaging members/engaging portions mounted/formed thereon, such as projections/recesses, bores etc., which have no inherent locking function, i.e. which are not directly located within the locking force flux but operate/actuate the movable locking element of the respective pair of locking elements for locking/unlocking. In this way, the operating strip needs to be dimensioned merely in terms of the operating function, as per se it does not have to absorb any locking forces.

In accordance with an advantageous embodiment which optionally will have to be claimed separately, the sterile container includes two operating levers each of which is coupled to a plurality of (more than one) locking elements and which jointly enable two-handed operation for opening and closing the sterile container. Said two operating levers are preferably arranged on opposite sides of the sterile container.

According to another advantageous embodiment which optionally will have to be claimed separately, the (at least one) operating lever is designed so as to exceed an eccentric point when it is brought to a locking position, thus preventing the at least one operating lever from inadvertently and automatically springing open into an opening position (self-locking).

According to another advantageous embodiment which optionally will have to be claimed separately, at the end opposed to the (at least one) operating lever of the operating strip coupled thereto an elastic tension element, preferably a spring, is provided which is preferably fastened on the sterile container (cover or trough). The tension element serves, during opening of the cover of the sterile container or shifting the operating lever to open, for assisting the locking elements which are forced back by the operating strip via the (at least one) operating lever in returning to the opening position. In addition, the tension element reduces the play inside the locking mechanism of the sterile container.

The locking elements may preferably be hook-shaped or claw-shaped pivot elements, movable locking pins or movable mushroom-type locking bolts. Upon locking by pivot elements, tension (or pressure) is applied by operating the operating lever(s) in the locking position to the operating strip coupled thereto and extending along the container or cover/door periphery, thus causing the pivot elements supported on the container trough or on the cover to be pivoted so that they engage in corresponding counter-pieces such as an eyelet, a latch or similar undercut, which causes the sterile container to be locked. When mushroom-type locking bolts or locking pins are used as locking elements, the operating strip advantageously includes at least one wedge-shaped crank or bulge which, when tension/pressure is applied to the operating strip, moves along the longitudinal direction of the operating strip, thus causing a close locking pin/mushroom-type locking bolt to be displaced in a direction extending substantially at right angles with the longitudinal direction of the operating strip, until the locking pin/mushroom-type locking bolt is in mesh with a corresponding counter-piece such as a recess or an eyelet.

In accordance with another advantageous embodiment which optionally will have to be claimed separately, the operating strip substantially follows the contour (periphery) of a cover/door of the sterile container. In other words, the operating strip extends at least in portions substantially along the outer edge or the inner edge of the cover of the sterile container. In yet other words, the operating strip extends along the inner surface of the container cover facing the container trough of the sterile container.

In accordance with another advantageous embodiment which optionally will have to be claimed separately, a one-piece operating strip is provided which runs substantially around the entire periphery of the cover of the sterile container. In other words, such flexible operating strip forms an (open) ring which runs around the periphery of the cover of the sterile container. The operating strip may be a cable pull, a rod system and/or a flat strip.

Alternatively, also plural operating strips may be provided which are circumferential over the periphery of the cover of the sterile container for a respective defined portion. For example, two operating strips each of which runs around a respective half of the cover of the sterile container may be provided.

In accordance with another advantageous embodiment which optionally will have to be claimed separately, the operating strip is preferably supported and/or guided at the corners of the cover via bearing elements on the sterile container (trough or cover). This facilitates the operation of the operating lever.

In accordance with another advantageous embodiment which optionally will have to be claimed separately, the operating strip serves exclusively for operating the locking elements coupled to the operating strip. This is to say that the operating strip has no other secondary function apart from this primary function of operating the locking elements.

In accordance with another advantageous embodiment which optionally will have to be claimed separately, the operating strip, too, is non-releasable on the container cover or the container trough. In this way it is achieved that, even in the case of repeated opening and closing of the sterile container, the operating strip does not detach from the latter and therefore is protected against damage, which helps to increase the service life of the sterile container.

In accordance with another advantageous embodiment which optionally will have to be claimed separately, individual locking elements of each pair of locking elements include internal locking members which are supported to be movable and/or pivoting on the container trough or the container cover and which are operatively engaged in the operating strip (2) for actuating movement/locking/unlocking.

Each locking element may include one or more locking members. Operative engagement in this context may be understood to be the fact that the respective locking members are not fixed to the operating strip but are merely operable by said operating strip. In other words, the operating strip is only adapted to act on the locking members in an operating manner without any locking members being tightly connected to the operating strip.

### BRIEF DESCRIPTION OF THE DRAWING FIGURES

Further advantages and features of the present invention will be evident from the description of preferred embodiments with reference to the Figures, wherein:

FIG. 1 shows a sterile container according to the invention comprising two operating levers and a closed operating strip;

FIG. 2 shows a sterile container according to the invention comprising an operating lever and a closed operating strip;

FIG. 3 shows a sterile container according to the invention comprising two operating levers and two operating strips;

FIG. 4 shows the insertion of the guideway for the operating strip within the seal of the sterile container; and

FIGS. 5a-c show various types of locking elements.

### DETAILED DESCRIPTION

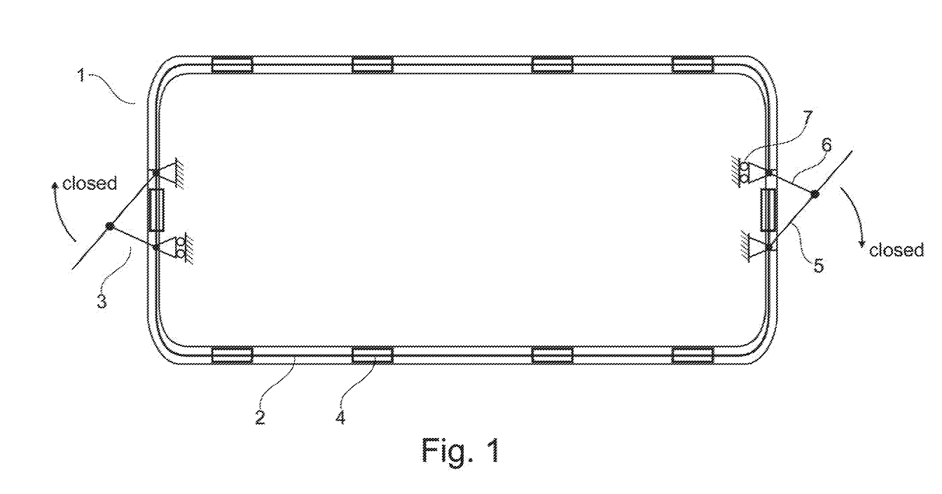

FIG. 1 illustrates a sterile container 1 comprising (active) discharge of concentrate having a plurality of locking elements 4 operable via two operating strips 2 by means of two common operating levers 3. Even if here a sterile container comprising active discharge of condensate is exemplified, the invention may as well be applied to any other sterile container.

More concretely speaking, the sterile container 1 includes a container trough 1a and a container cover 1b which can be locked to each other by means of at least one, presently two closing or locking mechanisms, each consisting of (at least) one operating lever 3, one operating strip 2 coupled to the operating lever 3 as well as plural locking elements 4 coupled to the operating strip 2. That is, plural locking elements or locking units 4 spaced apart along the periphery of the container cover (or door) are coupled or operatively coupled via a common operating strip 2 as force transmission means to a respective operating lever or operating unit 3.

In this special embodiment, the two operating strips 2 are combined by end-side overlapping or by appropriate telescopic coupling members to form a closed ring which runs around the entire periphery of the cover of the sterile container 1 and substantially follows the contours thereof. The two operating strips 2 are preferably continuously under tension both during the opening operation and during the closing or locking operation of the sterile container; therefore, alternatively to the operating strips, also cable pulls e.g. made from a wire rope may be used.

Each of the operating levers 2 has two arms 5, 6 one 5 of which is tightly connected to the sterile container or to the end side of the one operating strip 2 and the other 6 is movably connected to the end side of the other operating strip 2 such that the operating strips 2 are supported in the area of the articulation points with the arms 5 and 6, respectively, by appropriate bearings 7 on the container cover or on the container trough so that they can be displaced in the longitudinal direction via the operating levers 3 without detaching from the container cover or from the container trough.

When one of the operating levers 3 is actuated or operated, the movably supported arm of the operating lever 3 moves and thus permits the other arm of the operating lever to be folded over, wherein the two operating strips 2 move in the longitudinal direction relative to each other. Advantageously, each operating lever is dimensioned so that upon folding over the one arm 5 of the operating lever an eccentric point is exceeded, thus preventing the operating lever 3 from inadvertently automatically springing open (self-locking in the closing position).

The function of the locking mechanisms according to FIG. 1 can be described as follows:

In order to lock the container cover with the container trough, the two operating levers 3 provided on opposite sides of the sterile container are folded over serially or in parallel into the closing position. In so doing, both operating strips 2 are moved toward each other in the area of their end-side overlaps or telescopic coupling members so that the locking elements 4 coupled to the respective operating strip are jointly operated. If the one arm 5 of each operating lever 3 is fixedly articulated to the container cover or container trough, each operating lever acts independently and separately upon an operating strip 2. If the one arm 5 of each operating lever 3 is articulated to one of the operating strips and the other arm 6 is articulated to the respective other operating strip, however, each of the two operating levers acts upon each of the operating strips to which a plurality of locking elements is coupled.

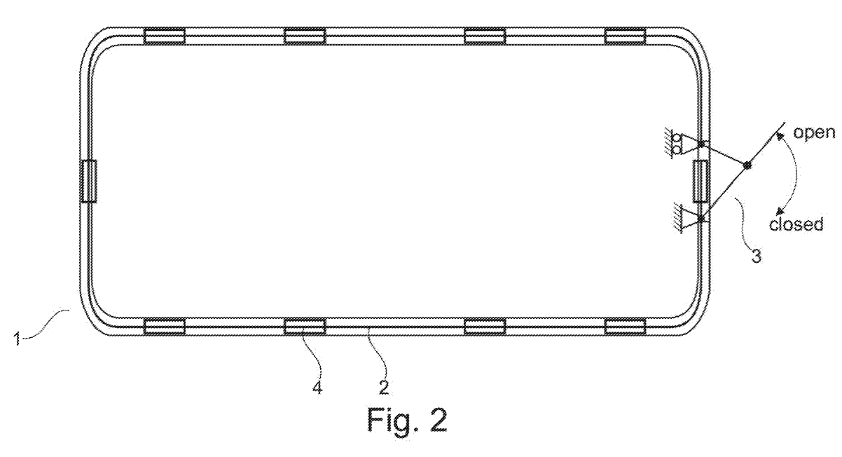

FIG. 2 illustrates a sterile container 1 according to the invention comprising only one operating lever 3 and only one operating strip 2. Like reference numerals denote like or similar components as in FIG. 1.

In this embodiment, too, the operating strip 2 is in the form of a flat strip or a rod system which substantially spans around the entire periphery of the container cover. Since the operating strip 2 is preferably continuously under tension both during the opening operation and during the closing or locking operation of the sterile container 1, as an alternative to the rod system also a cable pull, e.g. made from a wire rope, may be used.

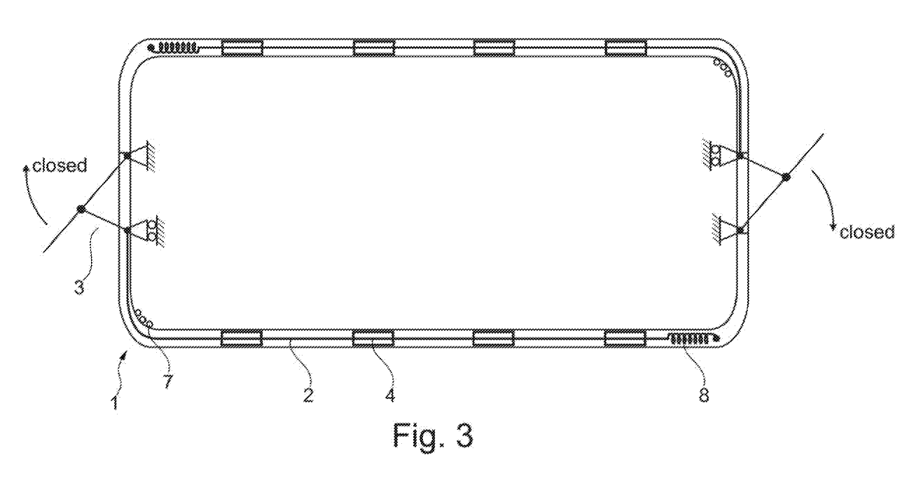

In this embodiment, an arm 5 of the operating lever 3 is fixedly coupled to the container cover or the container trough and the other arm is articulated to one end of the operating strip 2. The other end of the operating strip 2 is preferably kept under tensile stress via a biasing spring mounted on the container cover or on the container trough (not shown in more detail in FIG. 3).

When the one operating lever 3 is thus folded over to the closing position, the free end of the operating strip 2 moves toward the biasing spring, wherein the spring is further tensioned. Accordingly, the locking elements 4 coupled to the operating strip 2 are actuated for locking the container cover to the container trough.

The sterile container 1 shown in FIG. 3 equally includes two operating levers 3 and two operating strips 2. Like reference numerals denote like or similar components as in the preceding Figures.

In this embodiment, the two operating strips 2 are configured separately from each other and each of them runs around only just half of the entire periphery of the cover of the sterile container 1. In this way, they form an open ring around the container cover. One operating strip 2 is associated with each operating lever 3. At their ends opposed to the respective operating lever 3 the operating strips 2 include a tension spring 8 in the form of a coil spring providing a counterforce to the force applied by folding over the respective operating lever 3 into a closing position, which counterforce assists the return of the locking elements 4 to an opening position and reduces the play between the lever and the operating strip as well as between the operating strip and the locking elements 4.

At the corners of the cover of the sterile container 1 the operating strips 2 are moreover supported via rollers 7 so as to facilitate operation of the locking elements 4 by way of the respective operating strip 2.

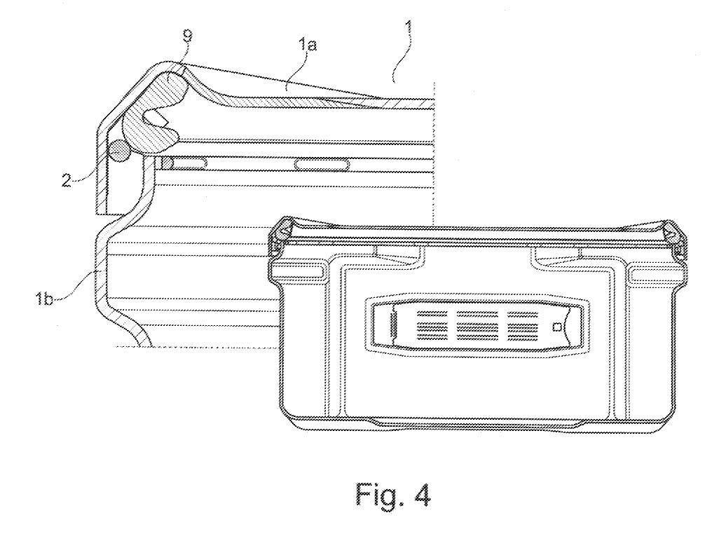

FIG. 4 illustrates a cross-sectional view of a sterile container 1 according to the invention from which the arrangement of the operating strip 2 relative to the cover 1a of the sterile container and of a seal 9 of the sterile container 1 is evident. The entire closing mechanism is provided outside the seal 9 and at no point does project into the interior of the sterile container 1. The seal 9 is disposed between the cover 1a and the container trough 1b of the sterile container 1. During the closing operation of the sterile container 1, the cover 1a is pulled onto the container trough 1b by means of rotatable latches, eccentrics or inclines as examples of the afore-mentioned locking elements 4. The seal 9 in this case constitutes the elastic part. The operating strip 2 in this embodiment is in the form of a rod system and/or of a cable pull, for example, and extends within a cavity formed by the seal 9 and the wall of the cover 1a. In other words, the seal 9 rests on the rim of the container trough 1b of the sterile container 1 and thus is located between the container trough 1b and the cover 1a of the sterile container 1. The operating strip 2 extends outside the container trough 1b (on the outside) in the area shielded by the container cover 1b projecting from the container trough 1a in a roof-like manner.

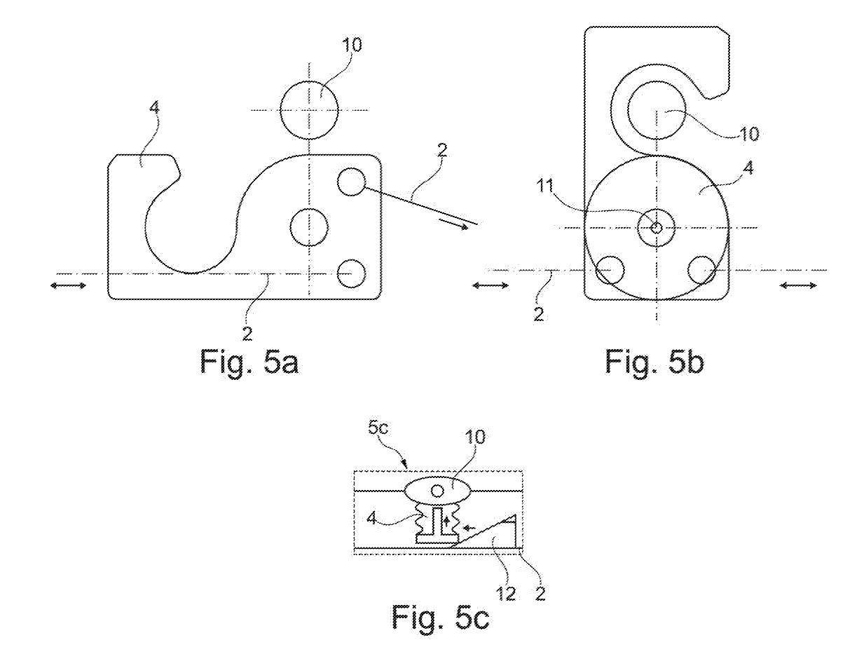

FIGS. 5a to 5b illustrate a locking element 4 in the form of a hook-shaped or claw-shaped pivoting element pivotally articulated to the container cover or the container trough which is pivoted from an opening position (FIG. 5a, the sterile container is opened and unlocked) to a locking position (FIG. 5b, the sterile container is closed and locked) by means of the operating strip 2. In this Figure, the operating strip 2 preferably is a cable pull. When operating the cable pull 2, the locking element in the form of the hook-shaped pivot element is pivoted about approx. 90° out of an unlocking position (cf. FIG. 5a) until in the locking position (cf. FIG. 5b) it is in mesh with a counter-piece 10 which in this embodiment is in the form of a pin. The locking element 4 in this embodiment is rotatably supported about a pivot point 11.

As an alternative and/or in addition, the sterile container 1 also may include at least one locking element 4 in the form of a locking pin as shown in FIG. 5c. Such locking element 4 substantially consists of a pin which is mounted to be axially movable on a platform or holder. A wedge-shaped crank or ramp 12 is provided at the operating strip 2. When the operating strip 2 is operated, the wedge-shaped crank 12 moves along the longitudinal direction of the operating strip 2 and beneath the platform/holder of the locking element/pin 4, thus causing the locking pin to be displaced to its axial direction which extends substantially at right angles with the longitudinal direction of the operating strip 2, until the locking pin is inserted in a counter-piece 10 in the form of an eyelet or a recess on the container trough or on the container cover (locking position). It is advantageous when such locking pin is spring-biased so that it will automatically return to an unlocking position when the operating lever is unfolded again.

In summary, the present invention relates to a sterile container comprising a plurality of locking elements that can be operated via at least one operating strip by means of at least one common operating lever. This allows active discharge of condensate and controlled pressure compensation in a reliable manner.

1. A sterile container comprising:
a container trough;
a container cover or door; and
a plurality of locking elements spaced apart from each other in a circumferential direction of the container cover and movably mounted on the container trough or on the container cover which can be engaged with at least one operating strip that comprises engaging portions/engaging members mounted or formed on the on the at least one operating strip and that is connected to at least one operating lever by which the plurality of locking elements can be centrally operated via the at least one operating strip so as to move the locking elements movably mounted on the container trough or on the container cover into a locking/unlocking position.

2. The sterile container according to claim 1, the at least one operating lever and the at least one operating strip that is engageable thereto and the locking elements to be brought/being in operative engagement therewith forms a locking mechanism, wherein two of said locking mechanisms are provided which, when connected in series, surround the periphery of the container cover in such manner that the two operating levers will be located on opposite sides of the sterile container so as to jointly enable a two-handed operation for unlocking and locking the sterile container.

3. The sterile container according to claim 1, wherein the at least one operating lever is adapted to exceed, when being moved to a locking position, an eccentric point, thus preventing the at least one operating lever from inadvertently springing open.

4. The sterile container according to claim 1, wherein, at the end of the at least one operating strip that is opposed to the at least one operating lever, an elastic tensioning element is provided which is connected to the at least one operating strip and to the container cover or the container trough so as to bias the at least one operating strip and thus the locking elements to be/being operatively engaged therewith into the unlocking position.

5. The sterile container according to claim 1, wherein the locking elements are hook-shaped or claw-shaped pivoting elements, locking pins or mushroom-shaped locking bolts.

6. The sterile container according to claim 1, wherein the at least one operating strip substantially follows the contour of the container cover of the sterile container.

7. The sterile container according to claim 1, wherein the at least one operating strip runs along substantially the entire periphery of the container cover of the sterile container.

8. The sterile container according to claim 1, wherein the at least one operating strip comprises a plurality of operating strips, each of the plurality of operating strips running along the periphery of the container cover of the sterile container for a respective defined section.

9. The sterile container according to claim 1, wherein the at least one operating strip is supported and/or guided at the corners of the container cover via bearing elements.

10. The sterile container according to claim 1, wherein the at least one operating strip is flexible.

11. The sterile container according to claim 1, wherein the at least one operating strip is adapted exclusively for operating the locking elements which can be/are operatively engaged with the at least one operating strip.

12. The sterile container according to claim 1, wherein the at least one operating strip is held to be non-releasable on the container cover or the container trough.

13. The sterile container according to claim 1, wherein the locking elements which can be/are operatively engaged with the at least one operating strip include locking members that are one of shiftably supported and pivotably supported on the container trough or the container cover and that, for actuating movement, can be/are operatively engaged with the engaging portions/engaging members at the at least one operating strip.
